(19) United States
(12) Patent Application Publication
(10) Pub. No.: US 2009/0311292 A1
(43) Pub. Date: Dec. 17, 2009

(54) PROCESS FOR SYNTHESIS AND INCORPORATION OF NITRIC OXIDE DONORS IN MACROMOLECULAR COMPOSITIONS

(75) Inventors: Ogari Pacheco, Nova Itapira (BR); Roberto Moreira, Itapira (BR); Amedea Seabra, Paulinea (BR); Gabriela Souza, Campinas (BR); Marcelo Oliveira, Campinas (BR)

Correspondence Address: BIRCH STEWART KOLASCH & BIRCH, PO BOX 747, FALLS CHURCH, VA, 22040-0747, US

(73) Assignees: CRISTALIA PRODUTOS QUIMICOS FARMACEUTICOS LTDA., Itapira (BR); UNIVERSIDADE ESTADUAL DE CAMPINASUNICAMP, Campinas (BR)

(21) Appl. No.: 12/441,341

(22) PCT Filed: Sep. 14, 2007
(86) PCT No.: PCT/BR2007/000236

§ 371 Date: Jul. 20, 2009

(30) Foreign Application Priority Data
Sep. 14, 2006 (BR) PI0603802-6
Aug. 21, 2007 (BR) PI0705221-9

(51) Int. Cl. A61K 9/00; A61K 38/06; A61P 17/02

(52) U.S. Cl. 424/400; 514/18

(57) ABSTRACT

The present invention describes a process for the synthesis of S-nitrosothiols and the subsequent incorporation of these compounds in hydrophilic macromolecular compositions. By the process described herein, the S-nitrosothiols are synthesized in a device (FIG. 2) in a first step from the S-nitrosation reaction of their respective precursor thiols (A), promoted by a mechanical action that puts the thiols in contact with the nitrous acid formed from nitrite anions in acidic medium (B), and in a second mechanical operation, the freshly formed S-nitrosothiols are incorporated in an application vehicle (C) based on hydrophilic macromolecular compositions that increases their thermal stability. Therefore, the process under consideration combine the pre-application synthesis of S-nitrosothiols with their subsequent incorporation in delivery vehicles, with provide a relative stabilization of the S-nitrosothiols for sufficient periods so that the formulations prepared by this process may be stored in a domestic refrigerator during its time of use in its several possible applications.

### FIELD OF THE INVENTION

This invention is situated in the field of devices for pre-application formulation of drugs and describes a device, and its variations, which allows the synthesis of S-nitrosothiols and their subsequent incorporation in hydrophilic macromolecular compositions, immediately prior to application.

### BACKGROUND OF THE INVENTION

Nitric oxide (NO) has been identified as the endothelium-derived relaxing factor responsible for the control of blood pressure (L J Ignarro, G M Buga, K S Wood, S Byrns, Endothelium-derived relaxing factor produced from artery and vein is nitric oxide. Proc. Natl. Acad. Sci. 84: 9265-9269 (1978) 1-4). Later, it has been found out that this diatomic molecule is also involved in neurotransmission, inhibition of platelet aggregation and immunological responses of a large number of pathological conditions. These findings have motivated an extensive research on the biochemical mechanisms involved in these actions and on exogenous sources of nitric oxide for biomedical applications. For example, the nitric oxide molecule produced in several cells of the human skin plays a key role in skin physiology and pathophysiology, regulating homeostasis and acting as a mediator of the cutaneous wound healing process.

The wound healing process is a complex physiological response intended to reestablish tissue integrity after a traumatic injury and involves the interaction between several cell types, extracellular matrix elements, cytokines and growth factors. Nitric oxide is one of the molecules synthesized endogenously at the site of injury through the action of the inducible nitric oxide synthase (iNOS) enzyme. As some of the physiological functions of nitric oxide include vasodilatation, inhibition of platelet aggregation, reduction of leukocyte cell adhesion and promotion of vascular smooth muscle cell proliferation, it is considered that nitric oxide plays an important role not only on the wound healing process, but also on the immunomediator responses of dermatological and inflammatory diseases.

In addition, nitric oxide exerts a powerful cytostatic and cytotoxic action against several intracellular pathogenic agents, such as Trypanosoma cruzi parasites responsible for malaria and leishmaniasis. The involvement of nitric oxide in the intracellular elimination of Leishmania by macrophages is well demonstrated (W Solbach, T Laskay. The host response to Leishmania infection. Adv Immunol. 2000; 74:275-317).

Therefore, the development of biomaterials that allow the topical or transdermal application and release of nitric oxide to leishmaniasis cutaneous ulcers is of great interest. Likewise, the localized release of nitric oxide may provide beneficial results in ischemic tissues by the increase of the local blood flow and stimulation of angiogenesis. Furthermore, the localized application of formulations capable of releasing nitric oxide or nitric oxide donors to target tissues may be used to promote transdermal absorption of other drugs as well as to attain the beneficial effects of NO at the site of application, thus avoiding the occurrence of undesirable side effects resulting from systemic administration. Therefore, there is a great interest in the development of NO-releasing systems.

As under environmental conditions, NO is a gas and reacts rapidly with the oxygen in the air, its localized and controlled release may be obtained with the use of substances that present a NO molecule chemically bound to its structure and that are able to donate this NO to other recipient molecules in the tissues or blood. Several NO-donor molecules are currently in clinical use, among which are the organic nitrates and nitrites, and sodium nitroprusside. However, the clinical applications of these classic NO-donors have limitations. The prolonged administration of sodium nitroprusside and organic nitrates may cause cyanide poisoning and vascular tolerance, respectively (Joahanning R J, Zaske D E, Tschida S J, Johnson S V, Hoey L L, Vancebryan K. A retrospective study of sodium-nitroprusside use and assessment of the potential risk of cyanide poisoning. Pharmacotherapy 1995; 15:773-777; Shishido S M, de Oliveira M G. Photosensitivity of aqueous sodium nitroprusside solutions: Nitric oxide release versus cyanide toxicity. Progress in reaction kinetics and mechanisms 2001; 26:239-261; Thadani U. Prevention of nitrate tolerance with angiotensin II receptor type 1 blocker in patients with stable angina: Yet another failed strategy to prevent tolerance. Cardiovascular drugs and therapy 2004; 18:339-342). In addition, these drugs have a weak antiplatelet action in therapeutic concentrations.

Another class of nitric oxide donors comprises the S-nitrosothiols (RSNOs), such as, S-nitrosoglutathione (GSNO) and S-nitrosoalbumin, which have already been identified as endogenous nitric oxide carriers and releasers in mammals.

The S-nitrosothiols present all physiological actions of free NO, such as, vasodilatation and inhibition of platelet aggregation, and have been subject of several studies and pharmacological strategies referring to the importance of nitric oxide in living systems. In the S-nitrosothiols, the nitric oxide is covalently bound to a sulfur atom through the —CSNO group and may be released by the homolytic or heterolytic cleavage of the S—N bond. The nitric oxide released in this way may be transferred to specific receptors such as enzymes containing iron atoms, to which nitric oxide may coordinate as a ligand (nitrosylation reactions) or proteins containing thiol groups (SH), to which nitric oxide may bound as an nitrosonium ion (NO⁺) in transnitrosation reactions (Carvalho-Filho, Ueno M, Hirabara S M, Seabra A B, Carvalheira J B C, de Oliveira M G, Velloso L A, Curi R, Saad M J A. S-nitrosation of insulin receptor, insulin receptor substrate-1 and protein kinase B/Akt: a novel mechanism of insulin resistance. Diabetes 2005; 54:959-967).

The possible reactions that the S-nitrosothiols may undergo in the biological environment are: thermal or photochemical decomposition with release of free NO, transnitrosation reactions and S-thiolation reactions (Hogg N. Biological chemistry and clinical potential of S-nitrosothiols. Free Radical Biology and Medicine 2000; 28:1478-86).

The S-transnitrosation reaction may be defined as the transference of the nitroso functional group from a RSNO to a thiol residue (RSH), as displayed in the following equation:

RSNO+R′SH=RSH+R′SNO

where R represents the organic radical of the S-nitrosothiol and R′ represents the organic radical of the nitrosated substrate. This reaction occurs by the nucleophilic attack of the thiolate anion on the nitrogen of the RSNO molecule. As the products formed in this reaction are also RSNO and RSH molecules, the reaction is reversible (Hogg N. The kinetics of S-transnitrosation—A reversible second-order reaction. Analytical Biochemistry 1999; 272:257-262). The S-transnitrosation reaction is of paramount importance from a biological standpoint because it allows NO transference from one species to another within the cells, representing an important mechanism of modification of protein activity. The S-nitrosation reaction represents a new mode of cell control and signalization. The transference of the nitrosyl residue from one thiol to another has been suggested as the mechanism of signalization by which nitric oxide controls the cell processes.

The inhibition or activation of enzyme activity by posttranslational S-nitrosation of cysteine residues of proteins has been acknowledged as an important cell signalization mechanism. Several proteins containing cysteine residues, including enzymes, ionic channels and transcription factors, have been proved capable of being S-nitrosated and having their functions altered. A number of examples of enzymes that had their activities modified due to the transnitrosation reaction may be mentioned, including creatine kinase, several caspases and insulin receptor substrates.

The S-nitrosothiols present as promising drugs for attaining the pharmacological effects of nitric oxide, without the inconveniences of the toxic action of sodium nitroprusside or development of tolerance to nitroglycerine and other organic nitrates.

Nevertheless, the S-nitrosothiols are thermodynamically unstable (Wang P G, Xian M, Tang X P, Wu X J, Wen Z, Caj T W, Janczuk A J. Nitric oxide donors: Chemical activities and biological applications. Chemical Reviews 2002; 102: 1091-1134, Baciu C, Gauld J W. Assessment of theoretical methods for the calculation of accurate structures and S—N bond dissociation energies of S-nitrosothiols (RSNOs). Journal of Physical Chemistry A 107: 2003: 46; 9946-9952; Singh R J, Hogg N, Joseph J, Kalyanaramant B. Mechanism of Nitric Oxide Release from S-nitrosothiols. The Journal of Biological Chemistry 271; 1996; 18596-18603) and their potential use in diverse medical-hospital or pharmaceutical applications is limited because their transport and storage conditions demand constant refrigeration.

As an example of the thermal decomposition of the S-nitrosothiols, the findings of a previous study showed that aqueous solutions of the S-nitrosothiols S-nitrosocysteine, S-nitroso-N-acetylcysteine and S-nitrosoglutathione undergo spontaneous thermal decomposition at 25° C. in the dark at concentrations ranging from 0.1 to 61.0 mmol L⁻¹ within a follow-up period of 3.5 hours (de Oliveira M G, Shishido, S M, Seabra A B, Morgon N H. Thermal stability of primary S-nitrosothiols: Roles of autocatalysis and structural effects on the rate of nitric oxide release. Journal of Physical Chemistry A 106; 2002: 38; 8963-8970). This study demonstrates that the storage of S-nitrosothiol solutions at room temperature for application purposes is not viable.

The thermal stability of the S-nitrosothiols can be increased by their incorporation in hydrophilic polymeric matrices that reduce the velocity of nitric oxide release through the cleavage of the S—N bond. Previous studies have demonstrated that the incorporation of S-nitrosothiols in liquid and solid polymers and in hydrogels promotes an stabilizing effect on the S-nitrosothiols, compared to what is observed in solution, thus improving their perspectives of use in topical or transdermal applications (patent applications PI 0004238-2, PI0201167-0 and PI0201168-9; Seabra A B, Fitzpatrick A, Paul J, De Oliveira M G, Weller R. Topically applied S-nitrosothiol-containing hydrogels as experimental and pharmacological nitric oxide donors in human skin. British Journal of Dermatology. 2004; 151 (5): 977-983; Seabra A B, de Oliveira M G. Poly(vinyl alcohol) and poly(vinyl pyrrolidone) blended films for local nitric oxide release. Biomaterials. 2004; 25 (17): 3773-3782; Seabra A B, da Rocha L L, de Souza G F P, Eberlin M N, de Oliveira M G. Photochemical and thermal nitric oxide release from S-nitrosoglutathione incorporated in poly(ethylene glycol)/H₂O matrix. Nitric Oxide-Biology and Chemistry. 2004; 11 (1): 54-54; Seabra A B, Da Rocha L L, Eberlin M N, De Oliveira M G. Solid films of blended poly(vinyl alcohol)/poly(vinyl pyrrolidone) for topical S-nitrosoglutathione and nitric oxide release. 2005; Journal of Pharmaceutical Sciences 94 (5): 994-1003).

The stabilizing effect produced by a polyethylene glycol (PEG) polymeric matrix on the thermal and photochemical decomposition of S-nitroso-N-acetylcysteine and S-nitrosoglutathione is significant in relation to the aqueous solution, both in the dark and under irradiation with visible light. However, the S-nitrosothiol continues to decompose in this matrix (Shishido S M, de Oliveira M G. Polyethylene glycol matrix reduces the rates of photochemical and thermal release of nitric oxide from S-nitroso-N-acetylcysteine. Photochemistry and Photobiology; 2000; 71(3): 273-280; Seabra A B, de Souza G F P, da Rocha L L, Eberlin M N, de Oliveira M G S-nitrosoglutathione incorporated in poly(ethylene glycol) matrix: potential use for topical nitric oxide delivery. Nitric Oxide-Biology and Chemistry. 2004; 11 (3): 263-272).

Likewise, the incorporation of the S-nitrosothiols, S-nitrosoglutathione (GSNO) and S-nitroso-N-acetylcysteine (SNAC) in poly(ethylene oxide)-poly(propylene oxide)-poly(ethylene oxide) (PEO-PPO-PEO) hydrogel reduces the rate of thermal and photochemical decomposition of RSNOs in relation to the aqueous solution. However, the RSNOs continue to decompose in this matrix. The initial rates of thermal decomposition of GSNO and SNAC incorporated in PEO-PPO-PEO gel at 37° C. were 2.3 and 7.2 μmolL⁻¹ min⁻¹, respectively (Shishido, S M, Seabra, A B, Loh, W, de Oliveira, M G. Thermal and photochemical nitric oxide release from S-nitrosothiols incorporated in pluronic F127 gel: potential uses for local and controlled nitric oxide release. Biomaterials; 2003: 24 (20): 3543-3553).

Such results demonstrated that the polymeric matrices exert a partial stabilizing effect reducing the rate of thermal decomposition but not preventing its continuity at room temperature. In this way, the hydrophilic polymeric matrices may allow the use of S-nitrosothiols immediately after preparation of their formulations, for periods that may be sufficient to obtain their pharmacological actions, without appreciable decomposition, but do not allow the stabilization of these formulations for prolonged periods during their transport and storage.

Several documents report approaches for the topical application of nitric oxide donors, many of them related to the incorporation of nitric oxide donors in polymeric matrices.

The patent application WO 99/38472 describes gels containing the following NO generators: nitroglycerin, nitroprusside, sodium nitrate, isosorbide dinitrate, L-arginine, pentaerythritol tetranitrate, mannitol hexanitrate and/or their analogues for topical applications for stimulation of the blood flow, blood vessel dilatation and treatment of vascular insufficiency. These molecules act as indirect NO generators. Acrylate is the polymer referred to in the formulations and the solvent is propylene glycol.

The U.S. Pat. No. 6,579,543 describes a preparation of a composition for dermatological application to obtain analgesic and antiinflammatory activities from formulations containing at least one antioxidant agent, at least one compound with anti-neuralgic action and at least one compound capable of promoting blood circulation, such as L-arginine, which induces endogenous NO production, and at least one compound with anti-depressive activity. In this patent, NO is generated in an indirect manner, that is, by the physiological action of L-arginine.

The patent WO 2002/34303 describes methods for treatment of vascular diseases characterized by NO insufficiency, such as Raynaud's syndrome, employing transdermal release patches. The formulations contain at least one antioxidant agent and at least one NO donor, for example, isosorbide dinitrate and isosorbide mononitrate, and/or at least one nitrosated inhibitor of the enzymatic conversion of angiotensin, one nitrosated calcium channel blocker, one nitrosated endothelial antagonist, one nitrosated angiotensin II receptor antagonist, and one nitrosated renin inhibitor.

The U.S. Pat. No. 6,747,062 describes the promotion of cutaneous wound healing of injured tissues (e.g., muscles, tendons, ligaments, skin, mucosas, bones and corneas) by tissue exposure to the presence of nitric oxide. The NO donors may be a NONOate, to increase NO concentration in the tissue, and monomethyl arginine, to reduce NO concentration in the tissue, and therefore adjust NO concentration in the tissue to the desired condition.

The patent application WO 2000/12112 describes a new coverage for the treatment of injuries in humans and animals composed of a substrate and the enzyme xanthine oxidase for enzymatic production of free NO.

The patent applications US2003165578, US2004009238 and US2002138051 describe methods and devices for the release of gaseous NO directly on wounds in mammals for promotion of cutaneous wound healing. In these documents, polymers are not used as NO carrier vehicles.

The documents WO 2003/063923 and US 2002122771 describe the preparation of hydrogels for covering of ulcers and wounds in topical applications on skin and bone cavities, with cuts, abrasions, surgical incisions or ulcers, by means of the in situ application of a liquid composition that is directly sprayed on the injured area. The mentioned active agent is any substance capable of releasing NO at the site of the wound.

The patent application WO 2002/17880 describes the preparation of biodegradable hydrogels, in which —SNO and/or —NNO groups are covalently bound to the polymeric chain to provide local release of NO.

The patent application US 2004259840 describes the preparation of NO-releasing compositions from lipid molecules with thiol, amine or alcohol groups containing nitroso residues, as NO-releasing systems for the treatment of atherosclerosis, cancer, eczema and arthritis.

The patent application WO 2003/049593 describes a composition for topical use composed of NO, its donor or pro-drug for prevention of necrosis, the drug being nitrosylated polythiolated cyclodextrin, nitrosylated polymer or long-life coating gel (for example, cyclodextrin). The vasodilating composition contains alkyl nitrite and S-nitrosothiol (preferentially NO— cyclodextrin) or nitrosyl metallic complexes. The composition may be administered topically, orally, locally or may be inhaled.

None of the approaches described above offers a solution to the problem of stability of the nitrosothiols, presented as either solutions or polymeric matrices, which is a key characteristic to make feasible the topical or transdermal use of S-nitrosothiol-containing formulations in medical, pharmaceutical or cosmeceutical applications.

On the other hand, there are documents that describe devices of pharmaceutical use intended to prepare a formulation immediately before use.

The U.S. Pat. No. 4,479,578 refers to a syringe-shaped or ampoule-shaped receptacle constituted of more than one compartment. One of the compartments containing a solid pharmaceutical product is temporarily isolated from another compartment containing an aqueous solution and the content of both compartments may be promptly mixed at the moment of application. This type of receptacle is intended to ready-to-use pharmaceutical products (active ingredient) that should be solubilized in an aqueous solution at the moment of application. In this patent document, the inventor does not mention the use of S-nitrosothiols in the compartments and, in addition, if such approach were extrapolated to the present invention, it would limit the transport and storage of the receptacles containing S-nitrosothiols exclusively under refrigeration conditions.

The document WO 2005/030111 refers to a device to treat wounds that comprises a receptacle with two or more separate compartments. The first compartment contains a first component and the second compartment contains a second component. The device is preferentially adequate for products used in wound healing treatment, specifically papain, in hydrogels comprising components that are unstable in their presence. The document does not mention the S-nitrosothiols as agents for wound healing treatment.

The U.S. Pat. No. 7,182,949 refers to a composition for topical application of an extemporaneous C vitamin preparation comprising a C vitamin precursor, except for esters, in contact with at least one enzyme that is capable of converting such precursor in C vitamin. This composition is intended to overcome the stabilization problems of C vitamin formulations by direct generation during or immediately before its application on the skin. This patent does not specifically describe a device but, in its examples, it explains, for instance, that both ingredients formulated in different emulsions should be stored in distinct compartments and mixed right before the application in order to allow the reaction of C vitamin formation to occur immediately before the topical application.

The document WO 2006/100155 refers to a device, with presentation form of bandage, intended to the treatment of wounds, which involves the use of nitric oxide. The device comprises the nitric oxide eluted in a polymeric matrix organized to remain in contact with the wound area, incorporated to a carrier material that regulates and controls the elution of the therapeutic dose of nitric oxide.

None of the above-mentioned devices is used in the extemporaneous synthesis of S-nitrosothiols from their precursors, by means of an S-nitrosation chemical reaction or a reaction of any other nature, occurred under specific concentration and pH conditions, and incorporation of the freshly prepared S-nitrosothiol in hydrophilic macromolecular compositions.

In view of the issues described above, it is notable the need of improvements in the art of making feasible the topical or transdermal use of formulations containing S-nitrosothiols in medical, pharmaceutical or cosmeceutical applications, preferably by means of approaches that allow the transport and storage of the components of the formulation, or their precursors, for prolonged periods at room conditions up to the moment of the first topical application and during the treatment.

In order to address this need, the present invention proposes a device, and its variations, which allows the synthesis of active S-nitrosothiols and their subsequent incorporation in hydrophilic macromolecular compositions, immediately before the topical application.

The devices described in the present invention offer an innovative solution to the transport and storage of S-nitrosothiol precursors and the formulation components at room temperature, and preparation of thermally unstable S-nitrosothiol formulations for medical, pharmaceutical or cosmeceutical applications.

The devices presented herein combine the pre-application synthesis of the S-nitrosothiols with their subsequent incorporation in delivery vehicles that yield a relative stabilization of the S-nitrosothiols for adequate periods in such a way that the resulting formulations in the device may be employed under room conditions in their several possible applications.

The field of application of the formulations prepared using the device of the present invention includes the stimulation of blood flow, blood vessel dilatation, treatment of vascular insufficiencies, treatment of Raynaud's syndrome, modification of skin pigmentation, promotion and acceleration of skin, muscle, tendon, ligament, mucosa, bone and corneal wound healing, prevention of necrosis, treatment de eczemas and arthritis, systemic lupus erythematosus and cutaneous leishmaniasis, among other applications.

### BRIEF DESCRIPTION OF THE INVENTION

The present invention describes a device, and its variations, which allows the synthesis of S-nitrosothiols and the subsequent incorporation of these compounds to hydrophilic macromolecular compositions, immediately prior to application.

In the devices described herein, the S-nitrosothiols are synthesized in a first step from the S-nitrosation reaction of their respective precursor thiols, promoted by a mechanical action that puts the thiols in contact with the nitrous acid formed from nitrite anions in acidic medium; in a second mechanical operation, the freshly prepared S-nitrosothiols are incorporated in a delivery vehicle based on hydrophilic macromolecular compositions that increase their thermal stability.

### BRIEF DESCRIPTION OF THE FIGURES

FIG. 1. Schematic presentation of a device developed according to the instructions of the present invention, which comprises two storage compartments and one reaction compartment, the formulation compartment that encloses the macromolecular matrix being uncoupled from the remainder of the device system.

FIG. 2. Schematic presentation of a device developed according to the instructions of the present invention, which comprises three compartments, the formulation compartment that encloses the macromolecular matrix being coupled to the remainder of the device system.

FIG. 3. Dermal blood flow after topical application of GSNO and SNAC in Pluronic F-127 hydrogels directly to the skin of healthy volunteers.

FIG. 4. (A) Percentage of retraction of the injured area due to wound treatment with topical application of GSNO incorporated to a hydrogel matrix and pure hydrogel (used as a control) 3 (d3), 5 (d5), 7 (d7), 14 (d14) and 21 (d21) days after injury. (B) Macroscopic detail of the wound area 5 (d5), 14 (d14) and 21 (d21) days after injury.

FIG. 5. Percentage of reepithelization of injured areas of animals treated with GSNO incorporated in a hydrogel and pure hydrogel (used as a control).

FIG. 6. Granulation tissue in control animals (A) and animals treated with GSNO (B) 21 days after injury (d21).

FIG. 7. Number of mast cells at the wound site in control animals and animals treated with GSNO 21 days after injury (d21).

### DETAILED DESCRIPTION OF THE INVENTION

The devices of the present invention allow the synthesis of S-nitrosothiols and their subsequent incorporation in hydrophilic macromolecular compositions, immediately prior to application.

According to the present invention, the device for the synthesis of S-nitrosothiols and incorporation in macromolecular compositions immediately before their application comprises storage compartments that enclose separately the precursor reagents for the synthesis of the S-nitrosothiol in the reaction compartment immediately before its incorporation in a macromolecular composition enclosed in a formulation compartment, which is either coupled to or uncoupled from the remainder of the device system, thus constituting alternatively a kit.

The device of the present invention allows the transport and storage of the S-nitrosothiol precursors and the components of the formulation at room temperature, and preparation of the formulation right before its application.

According to the present invention, the device for the pre-application synthesis of S-nitrosothiols and incorporation of the freshly prepared S-nitrosothiol in macromolecular compositions comprises:
(i) a reaction compartment containing an acid aqueous solution;
(ii) one storage compartment containing a mixture of a nitrosable thiol, or an acid salt of the nitrosable thiol, and a nitrite salt, both in the solid form, or optionally, two storage compartments to enclose separately a nitrosable thiol in the solid form and a nitrite salt in the solid form; and
(iii) a formulation compartment containing a hydrophilic macromolecular matrix or composition, which may be either coupled to or uncoupled from the reaction compartment,
   which, by means of a first mechanical action, promotes the contact of the nitrosable thiol and nitrite salt deriving from different storage compartments or from the same storage compartment with the acid aqueous solution enclosed in the reaction compartment, thus forming an S-nitrosothiol through an immediate S-nitrosation reaction, and by means of a second mechanical or transference action, the device promotes the incorporation of the freshly synthesized S-nitrosothiol in the macromolecular matrix, thus resulting in a pharmaceutical formulation in the form of a viscous solution or a hydrogel proper for use in topical medical or pharmaceutical applications.

According to the present invention, a nitrosable thiol is any molecule that contains one or more sulfhydryl groups (—SH) in its structure. The nitrosable thiols may be, more specifically, an amino acid, a peptide or a protein containing one or more sulfhydryl groups (—SH). Preferably, the nitrosable thiol employed in the device of the present invention should be selected from the group consisting of glutathione (GSH), N-acetyl-cysteine (NAC) and N-acetylpenicillamine, or their pharmaceutically acceptable salts. In the devices of the present invention, instead of a thiol, a mixture of nitrosable thiols may be used.

The acid aqueous solution may be constituted by a mineral acid, such as hydrochloric acid, or by an organic acid, such as citric acid. The concentration of the acid aqueous solution may range from 1 to 4 mol L⁻¹.

The macromolecular matrix or composition may be constituted of one or more biocompatible hydrophilic macromolecular components, and each component may either have or not tissue adhesion properties.

The macromolecular matrix can be, for example, poly(ethylene glycol) (PEG) in any of its commercially available presentations or triblock copolymer of poly(ethylene glycol)-poly(propylene glycol)-poly(ethylene glycol) (PEO-PPO-PEO) in any of its commercially available presentations, or hydroxyethyl cellulose (HEC) in any of its commercially available presentations, or hydroxymethyl cellulose (HMC) in any of its commercially available presentations, or Carbopol® in any of its commercially available presentations, or poly(vinyl alcohol) in any of its commercially available presentations, or poly(vinyl pyrrolidone) in any of its commercially available presentations.

In addition, any of the macromolecular components may already be crosslinked or undergo crosslinking by the action of a crosslinking agent, or any other substance used to improve the adhesion properties of the macromolecular composition.

The macromolecular composition can contain buffering, conserving, colorant, dispersing agents and metal complexants, or mixtures thereof. A dispersing agent, such as mannitol and/or citric acid, may alternatively be packed in the storage compartment of the device of the present invention.

In any variation of the device of the present invention, the mixture of the nitrite salt with the nitrosable thiol in acid medium leads to the formation of the corresponding S-nitrosothiols. In case the acidification of the nitrite solution is obtained by the presence of hydrochloride acid (HCl), and if the nitrite salt is a sodium salt, the following chemical equations describe the formation of nitrous acid and thiol nitrosation.

Na⁺+NO₂⁻+H⁺+Cl⁻→HO—NO+Na⁺+Cl⁻

RSH+HO—NO→RSNO+H₂O

where RSH represents the nitrosable thiol, HO—NO represents the nitrous acid originating from the dissolution of sodium nitrite in acid solution, and RSNO represents the S-nitrosothiol, which is the active principle of the formulations prepared with these devices. It should be noted that the formula HO—NO represents the associated nitrous acid and may also be represented as HNO₂.

The acid medium for thiol nitrosation may also be obtained from the dissolution of the thiol itself in water, if the thiol is used in the form of chloride, because the chlorides of the thiols under consideration are acid salts.

The amounts of nitrosable thiols and nitrite salts present separately or conjunctly in the storage compartments of the device in the solid forms should be equimolar or there may be a slight excess in the molar quantity of nitrite salt (approximately up to 10%) in relation to the molar quantity of thiol.

The concentrations of S-nitrosothiols incorporated in the delivery vehicles may range from 0.1 μmol L⁻¹ to 600 mmol L⁻¹. If the nitrite salt and the solid thiols are diluted in inert water-soluble diluents, nanomolar concentrations may be obtained in the incorporation of the synthesized S-nitrosothiols in the delivery vehicles based on the hydrophilic macromolecular compositions.

The formulation containing one or more S-nitrosothiols can be applied in the form of liquid solution or gel, or may jellify after contact with the target tissue or through a thixotropic activity inherent to the macromolecule.

The operation of the device of the present invention involves the following steps:

(a) to promote a first mechanical action in such a way that the nitrosable thiol and the nitrite salt are released from their respective storage compartments and get in contact with the acid aqueous solution in the reaction compartment, followed by manual agitation for approximately 5 seconds, allowing the occurrence of the S-nitrosation reaction;

(b) to promote a second mechanical action in such a way that the S-nitrosothiol freshly prepared in acid aqueous solution, flows off the reaction compartment to be incorporated in a macromolecular matrix, which increases the thermal stability of the active principle and acts as an application vehicle, followed by homogenization by manual agitation for approximately 10 seconds; and

(c) the formulation containing the S-nitrosothiol is ready to be topically applied to patient and, depending on the viscosity of the final formulation, the possible applications of the prepared formulation include: use of a spatula to remove the desired amount of the formulation from the compartment; adaptation of a device shaped as a spray, sprinkler or similar to the formulation compartment of the device, containing the freshly prepared formulation; or fabrication of the final compartment of the device shaped as a tube or syringe.

In the particular case of using PEO-PPO-PEO hydrogel as a macromolecular composition, the device should be stored under refrigeration in a domestic refrigerator (temperature around 5° C.) for approximately 30 minutes before operating the device. In this case, the decrease of the temperature allows that the PEO-PPO-PEO macromolecular composition is presented as a viscous liquid, facilitating the incorporation of the S-nitrosothiol and the homogenization of the final formulation. After incorporation of the S-nitrosothiol in the PEO-PPO-PEO macromolecular composition, the formulation can be maintained at room temperature, at which the composition will pass to a gel state. However, in order to prolong its shelf life, it is recommendable to maintain the prepared formulation stored in a domestic refrigerator.

Alternatively, the PEO-PPO-PEO matrix can be used as a viscous polymeric solution. The use of PEO-PPO-PEO viscous aqueous solution does not implicate in previous refrigeration of the device in a domestic refrigerator, prior to the mechanical actions. As the macromolecular composition of the PEO-PPO-PEO aqueous solution is less viscous than the solution that jellifies at room temperature (10% versus 30%, respectively), S-nitrosothiol incorporation and its homogenization in the polymeric solution is facilitated.

In all particular cases where the S-nitrosothiol is incorporated in the macromolecular composition after its synthesis in acid aqueous medium, the pH of the macromolecular composition can be adjusted with the presence of buffering salts in order to achieve the desired final pH for the formulation, for example, within the 5.5 to 7.4 range.

The devices in all variations described in this patent application can be transported and stored at room temperature, as the stability of thiols and nitrite salt, particularly sodium nitrite, solid and dry, packed separately or conjunctly, is relatively high at room conditions, differently from the S-nitrosothiols that are unstable.

The formulations prepared with this device may be used for stimulation of blood flow, blood vessel dilatation, treatment of vascular insufficiencies, treatment of Raynaud's syndrome, modification of skin pigmentation, promotion and acceleration of skin, muscle, tendon, ligament, mucosa, bone and corneal wound healing, prevention of necrosis, treatment de eczemas and arthritis, systemic lupus erythematosus and cutaneous leishmaniasis, among other applications.

A detailed description of the present invention will be featured below as illustrative examples, which are not a limitation upon the scope of the present invention.

### Example 1

### Device for the Pre-Application Synthesis of S-Nitrosothiols and Incorporation in a Macromolecular Matrix, in which the Formulation Compartment is Uncoupled from the Reaction Compartment

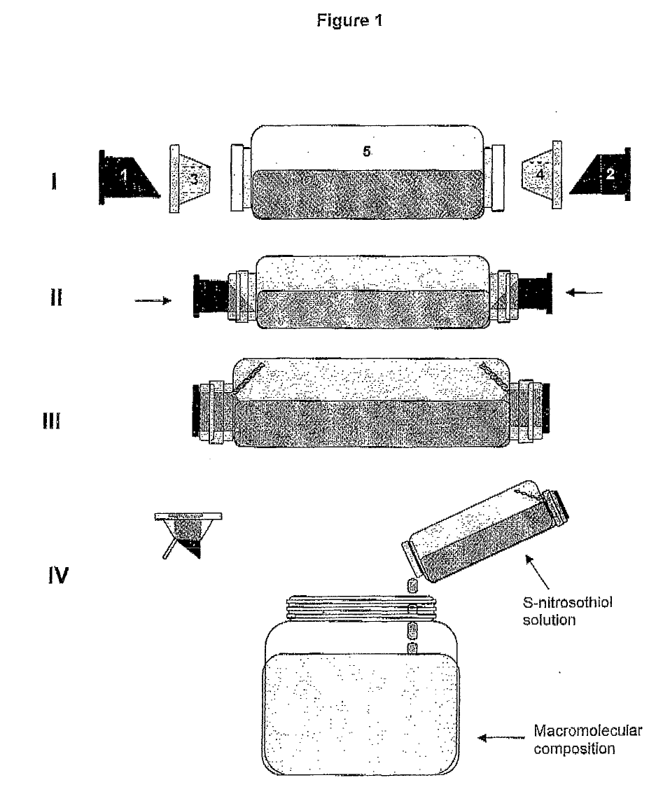

A schematic presentation of a possible device prepared according to the instructions of the present invention is illustrated on FIG. 1. Panel I of FIG. 1 displays the integrating components of a device that contains two storage compartments [(a)+(c)] and [(b)+(d)] and a reaction compartment (e). The components (a) and (b) enclose, separately, the nitrosable thiol and the nitrite salt, both in the solid and dry form, while the components (c) and (d) have cup-shaped format and have the function of wrapping the components (a) and (b) in a way to isolate the storage compartments from the reaction compartment (e), which encloses an acid aqueous solution. The components (a) and (b) have a sharp format in their open end towards the base of the components (c) and (d), which will be disrupted as the device is put in operation. In this Example, the storage compartments are located at the end of a vial that represents the reaction compartment.

Panel II of FIG. 1 displays the device constituted by 3 compartments mounted and ready to operate. Device operation will occur by simultaneous pressing of both components (a) and (b) towards the reaction compartment (e) causing the rupture of the base of components (c) and (d).

Panel III of FIG. 1 displays the result of the simultaneous pressing of the components (a) and (b), indicating that, with this action, the nitrosable thiol and the nitrite salt get in contact with the acid aqueous solution in the reaction compartment (e), where, after agitation, the nitrosable thiol and the nitrite salt react instantaneously by S-nitrosation, obtaining the S-nitrosothiol of therapeutic interest, in an acid solution.

Panel IV of FIG. 1 displays the addition of the S-nitrosothiol solution to the formulation compartment, which encloses the macromolecular matrix and is uncoupled from the remainder of the device. This simple operation allows the formation of the final product that will be applied to the patient.

The horizontal format of the reaction compartment (e), as illustrated in FIG. 1, is not restrictive for the adequate performance of the present invention, and a vertical format of this compartment is also acceptable. Instead of being positioned at the ends of the reaction compartment, the storage compartments may optionally be positioned on the lateral walls of the reaction compartment.

In order to obtain 200 mL of a viscous solution of GSNO 200 uM from a device operating according to FIG. 1, the device comprises, for example, 12.3 mg of GSH and 170 mg of mannitol in the storage compartment (a), 3 mg of sodium nitrite and 180 mg of mannitol in the storage compartment (b), 2 mL of HCl solution 2.0 M in the compartment (e). As the device is operated, the synthesis of S-nitrosoglutathione occurs and the resulting solution containing S-nitrosoglutathione is transferred to a receptacle containing 198 mL of F127 solution (10.5% w/w) in phosphate buffer, pH 7.

### Example 2

### Device for Pre-Application Synthesis of S-Nitrosothiols and Incorporation in a Macromolecular Matrix in which the Formulation Compartment is Coupled to the Reaction Compartment

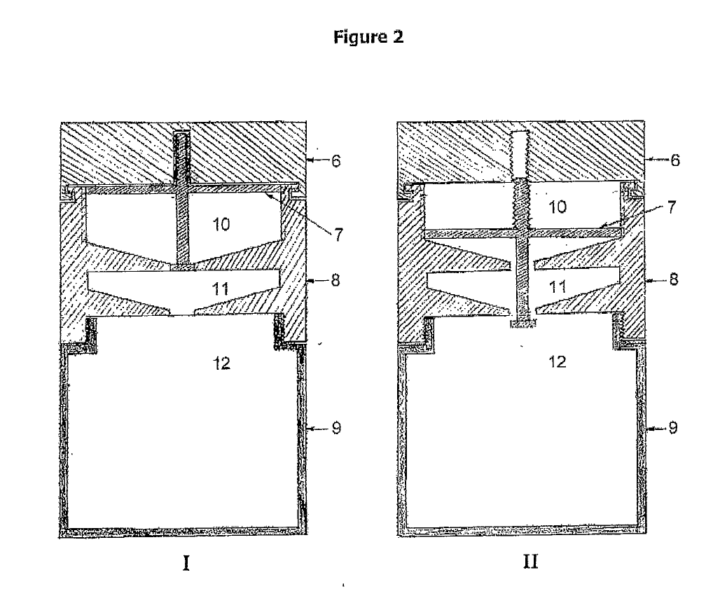

FIG. 2 displays an alternative for fabrication of the device of the present invention, in which the compartment that encloses the macromolecular matrix is coupled to the device, forming a single system.

The device illustrated in the panel I of FIG. 2 comprises four sections (1, 2, 3 and 4) and three compartments (A, B and C). In this case, compartment B is both the storage compartment (prior to device operation) and the reaction compartment (after device operation). Compartments A and C are the storage and formulation compartments, respectively. The section 1 is coupled to the section 3 by means of the notch represented between both sections. This notch allows that the section 1 rotates freely over section 3. Section 2 consists of a t-shaped piston with an upper screw threaded to section 1. The horizontal bar on section 2 consists of two pawls that insert bilaterally into two diametrically opposed grooves that run vertically on the internal wall of section 3. The notch on section 2 in these grooves is intended to avoid that section 2 rotates in relation to section 3. Section 3, in turn, is coupled to section 4 through a threaded connection. Section 3 posses two internal compartments (A and B) isolated from each other by a first septum on its central portion (compartment A septum). Compartment B is separated from the compartment C of section 4 by a second septum (septum of compartment C). Section 4 posses an internal compartment (C). Compartment A encloses an acid aqueous solution. Compartment B encloses a nitrosable thiol and nitrite salt mixture, both in solid and dry form. Compartment C encloses the macromolecular composition in the form of solution.

When section 1 is rotated against section 3, section 2 is pushed downwards by the counter-thread movement of the upper screw. As it is pushed downwards, section 2 breaks the septum of compartment A releasing the acid aqueous solution from compartment A to the compartment B. When the components of compartments A and B are mixed, an S-nitrosation reaction of the thiol will instantaneously occur under stoichiometric conditions. As the rotation movement of section 1 in relation to section 3 continues, section 2 will reach the septum of the compartment B and will cause its rupture, making that the solution of S-nitrosothiol freshly synthesized in the compartment B be added to the macromolecular matrix enclosed in the compartment C. After rupture of the septum of the compartment B, both pawls of section 2 will reach the inferior limit of the internal lateral notches of section 3, stopping the rotation of section 1 in relation to section 2. At this moment, as the operator undertakes a stronger rotation force on section 1 using one of the hands, while holding the inferior part (sections 3 and 4) of the device with the other hand, sections 3 and 4 will be disconnected at the threaded connection existing between them. This means that sections 1 and 3 can be removed as a single cover of section 4. After opening, section 4 may be covered with either a regular threaded cover, if the final formulation is a hydrogel, or a sprinkler or spray, if the final formulation is a solution. Both options of cover should be supplied in the device's package. After closure of section 4 with a regular cover, the vial should be agitated during 10 to 15 seconds before topical application of the S-nitrosothiol-containing macromolecular composition.

Panel II of FIG. 2 depicts the same device displayed on the panel I of FIG. 2, representing the section 3 in its lower position, after device operation and the consequent rupture of the septa of the compartments A and B. It should be noted that, in this situation, the inferior end of the vertical axis of section 2 should be situated below the base containing the septum of compartment B, in order to provide a space through which the solution in compartment B can flow over the macromolecular composition solution enclosed in compartment C.

Alternatively, the nitrosable thiol and the nitrite salt may be packed individually, separated by a horizontal division of compartment B, i.e., a septum that will also be disrupted with the dislodgment of section 2. In this case, the nitrosable thiol will be packed in the upper division of compartment B, which firstly will receive the acid aqueous solution. The nitrite salt will be packed in the lower division of compartment B, which will further receive the nitrosable thiol freshly solubilized in the acid aqueous solution, allowing the occurrence of the S-nitrosation reaction with immediate production of the desired S-nitrosothiol.

To prevent leakage and ensure the seal between sections 1 and 3 and between sections 3 and 4, it may be used joint rings or gaskets made from flexible polymeric materials compatible with the chemical nature of the reagents to be used and with the purposes of packaging of products for medical or pharmaceutical applications.

It should be emphasized that this device will work as described above only if it is operated in the vertical position represented in FIG. 2.

The materials that can be used in the fabrication of the devices presented as examples on FIGS. 1 and 2 include all rigid polymers compatible with the chemical nature of the reagents to be used and with the purposes of packaging of products for medical or pharmaceutical applications. Ideally, the polymeric materials to be used in the fabrication of the device should be light-proof in order to prevent the photodegradation of the components of the device.

### Example 3

### Stability of the Components Enclosed in the Compartments of the Device of the Present Invention

S-nitrosothiols, such as S-nitrosoglutathione and S-nitroso-N-acetylpenicillamine, are unstable in aqueous solution and are therefore commercialized as dry powders with label information indicating that the products should be stored under refrigeration (0° C. for S-nitrosoglutathione and −20° for S-nitroso-N-acetylpenicillamine). Like S-nitrosothiols, thiols, such as glutathione and N-acetylcysteine, are unstable in aqueous solution and are therefore commercialized as dry powders with label information indicating that the product should be stored under refrigeration (2-8° C.).

This knowledge indicates that the use of thiols and S-nitrosothiols in solution in the compartments of the devices of the present invention is not viable.

The use of commercially available solid presentations of S-nitrosothiols in one of the device's compartments with the sole purpose of yielding pre-application solubilization and incorporation of the S-nitrosothiol would limit the transport and storage of the device at temperatures below 0° C. because the S-nitrosothiols are unstable at room temperature, as demonstrated by the stability assay exemplified for S-nitrosoglutathione (GSNO).

Samples of solid GSNO were stored in amber glass vials and maintained at room temperature (25° C.) for evaluation of its stability. GSNO content in the samples at day 0, after 48 hours (day 2) and after 5 days was quantified from the absorbance readings of the aqueous solutions 500 μmol L⁻¹ at 336 nm. Table 1 displays the results of the GSNO percent content calculated from the differences in the absorbance readings at day 0 and at days 2 and 5. According to the results, after 48-hours of storage at room temperature, decomposition of approximately 52% of the initial GSNO content occurs.

TABLE 1
Stability of S-nitrosoglutathione under storage
conditions at room temperature (25° C.).

| | Content (%) | | |
|---|---|---|---|
| | | After 2 | After 5 |
| Substance | Initial (day 0) | days | days |
| GSNO | 100 ± 6 | 48 ± 6 | 36 ± 6 |

The viability of the storage of thiols, precursors of S-nitrosothiol, in the solid form and at room temperature in the compartments of the devices of the present invention was confirmed by the stability assay.

Samples of solid glutathione (GSH) were stored in amber glass vials and maintained at temperatures of 30° C. and 40° C. for evaluation of its stability. GSH content in the samples was determined by UV-visible spectrophotometry from the reaction between GSH and NaNO₂ in acid medium (aqueous HCl solution, 2.0 mol L⁻¹) forming GSNO with absorption bands at 336 nm and 545 nm. GSNO formation (0.05 mol L⁻¹) was quantified by the intensity of the GSNO absorption band at 545 nm. Table 2 presents the results of the GSH content after 165 days of storage at 30 and 40° C., demonstrating that GSH remains stable under these storage conditions. The stability of solid glutathione is not altered by the presence of dispersing mannitol, allowing the storage of these substances combined, as displayed on Table 2.

TABLE 2
Stability of glutathione (GSH) at different storage
temperatures.

| | | Content (%) | | | | |
|---|---|---|---|---|---|---|
| | T | | 30 | 70 | 105 | 165 |
| Sample | (° C.) | Initial | days | days | days | days |
| GSH | 30 | 100 ± 6 | 100 ± 6 | 102 ± 6 | 103 ± 6 | 100 ± 6 |
| | 40 | 100 ± 6 | 100 ± 6 | 100 ± 6 | 99 ± 6 | 101 ± 6 |
| GSH + | 40 | 102 ± 6 | 103 ± 6 | 96 ± 6 | | |
| mannitol | | | | | | |

In the solid form and at room temperature, stability assays show that GSNO is unstable, presenting a significant decomposition (approximately 50%) after 2 days under this condition, while GSH remained stable during at least 165 days of follow up by UV-visible spectrophotometry. The synthesis of the S-nitrosothiol prior to incorporation and application, as provided by the device of the present invention, is therefore, required. After device operation, the obtained S-nitrosothiol formulation should be maintained under refrigeration in a domestic refrigerator.

The sodium nitrite used in the device is already marketed in the solid form, not requiring refrigeration, inert atmosphere or light-proof containers. Therefore, the solid sodium nitrite is recognizably stable. However, sodium nitrite decomposes into an acid solution with N₂O₃ evolution.

The macromolecular composition in aqueous solution is stable for at least 2 years.

### Example 4

### Stability of the S-Nitrosothiols Prepared and Incorporated in PEO-PPO-PEO Matrix Using the Device of the Present Invention

Table 3 displays the results of the stability of the GSNO incorporated in PEO-PPO-PEO matrix (commercial brand Pluronic F-127) obtained using the device described in the present invention, at three different concentrations: 50, 100 and 200 μmol L⁻¹. To date, the gathered stability data make up a study duration of 110 days under refrigeration in domestic refrigerator (5-8° C.) and demonstrate that the GSNO incorporated in the PEO-PPO-PEO macromolecular matrix is relatively stable at all three tested concentrations presenting little decomposition (about 20% decrease in relation to the initial GSNO content), which does not compromise its application.

TABLE 3
Stability of the formulation containing S-
nitrosoglutathione (GSNO) stored under refrigeration in a
domestic refrigerator (5-8° C.).

| | |
|---|---|
| GSNO | |
| concentration | Content (%) |

| in a hydrogel | | 30 | 45 | 110 |
|---|---|---|---|---|
| formulation | Initial | days | days | days |
| 50 μmolL⁻¹ | 100 ± 6 | 96 ± 6 | 89 ± 6 | 85 ± 6 |
| 100 μmolL⁻¹ | 100 ± 6 | 103 ± 6 | 100 ± 6 | 80 ± 6 |
| 200 μmolL⁻¹ | 100 ± 6 | 90 ± 6 | 90 ± 6 | 85 ± 6 |

### Example 5

### Results of the Application of an S-Nitrosothiol-Containing Hydrogel Prepared Using the Device of the Present Invention on the Intact Skin of Healthy Human Volunteers

5.1. Volunteers. Seven healthy human volunteers (4 males; 3 females) were recruited. The study was approved by the Regional Ethics Committee (Lothian Regional Ethics Committee) from Scotland, where the experiments were undertaken. All volunteers signed an informed consent form. Smokers and individuals with dermatological diseases were excluded. The volunteers were prohibited of consuming caffeine for at least 12 hours before microdialysis.

5.2. Blood flow measurements. The device of the present invention was operated immediately after its refrigeration. The formulations resulting from device operation were F-127/GSNO and F-127/SNAC solutions, which jellified within approximately 5 minutes after application on the forearm skin of the volunteers due to temperature raise, forming F-127/GSNO and F-127/SNAC hydrogels. The concentration of S-nitrosothiol in the formulation is 0.3 molL⁻¹. Cutaneous vasodilatation, measured by means of red cell blood flow, was monitored by laser Doppler perfusion imaging (Moor Instruments Ltd) with a sensor connected to the skin, which allowed the simultaneous reading of blood flow from two laser guides. The 7-cm-diameter guides were placed on the volunteers' skin exactly on the site of application of the hydrogel. A perfusion monitor was connected to a personal computer and the vasodilatation readings were obtained continuously using specific software (moorLAB v1.31 for Windows© Moorsoft Instruments Ltd). At 10-minute intervals, new readings of mean vasodilatation were performed (n=3) within a 3-hour period. Hydrogel without S-nitrosothiol served as a control.

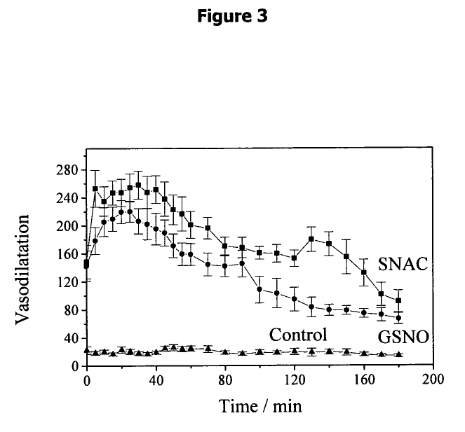

FIG. 3 shows the variations in blood flow as a function of time secondary to the topical application of a formulation prepared using the device of the present invention (0.3 molL⁻¹ of nitrosothiol in Pluronic F-127 24% m/m hydrogel), compared to the control. The results showed that the topical application of the hydrogels resulted in a 12-fold increase in local blood flow, in all volunteers, compared to the control. The maximum blood flow value was reached within 30 minutes, returning to the basal values after 3 hours.

### Example 6

### Cutaneous Wound Healing after Application of the Formulation Prepared Using the Device of the Present Invention

6.1. Acceleration of cutaneous wound healing in an animal model. In order to demonstrate the wound healing effect of RSNOs in the cutaneous wound healing in an animal model, GSNO was synthesized and incorporated in Pluronic F-127 hydrogel by operating the device described in the present invention. The formulation of freshly prepared GSNO (100 μmol L⁻¹) was topically applied to the wounds of the animals. Wistar rats (n=10) were housed in individual cages with free access to water. In the first day (Day 0-d0), an excisional wound (2×2 cm) was made on the back of the animals, under general anesthesia. The wound was covered with either pure hydrogel (control animals) or GSNO-containing hydrogel (treated group). Thereafter, the wounds were closed with a dressing. Daily, up to the fourth day after injury, the dressings were removed, and the wounds were gently cleaned with cold saline. The hydrogel (either containing GSNO or not) was applied and the dressing was replaced. From the fifth day after injury on, the wounds were no longer closed with a dressing.

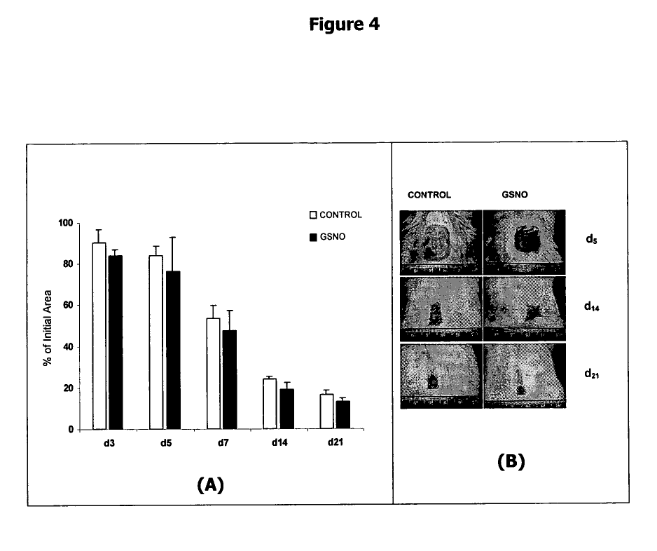

Wound retraction was measured and reepithelization was evaluated histologically. Wound contours were traced in a transparent paper sheet at the day of injury and after 3, 5, 7, 14 and 21 days. The tracing area was determined using image-analysis software (Image-Pro) and the results were expressed as percentages of the initial area. Blood pressure was measured at the beginning and end of the experiments. After euthanasia, a fragment containing the wound and the adjacent healthy skin was removed. The fragments were fixed in formalin solution, processed and embedded in paraffin. The paraffin-embedded specimens were serially sectioned and 5-μm-thick cuts were obtained and stained using the following techniques: hematoxylin-eosin (for overall observation of the tissue fragment), picro-Mallory (for observation of the connective tissue) and picrosirius red (for observation of the collagen fibers). The blood pressure of the control and treated animals was equivalent at the beginning and end of the experiments. FIG. 4 displays the retraction of the wound area in the animals treated with the GSNO-containing hydrogel and in the control animals (treated with pure hydrogel).

According to FIG. 4, after 3, 5 and 7 days of injury, wound retraction in the group treated with GSNO-containing hydrogel was greater than that observed in the control group (p=0.01; p=0.05; p=0.007, respectively). After 14 and 21 days of injury, neither of the groups presented blood clot and both exhibited decreased wound areas. However, the decrease of the wound area was more evident in the GSNO-treated group, compared to the non-treated control group. After 14 and 21 days of injury, there was new epidermis formation, which was more accentuated in the GSNO-treated animals, compared to the control animals. After 14 and 21 days of injury, wound contraction was greater in the animals treated with GSNO in relation to the controls. Fourteen days after injury, the wound area in the control animals was 22% larger than that of the GSNO-treated animals; twenty-one days after injury, the wound area in the control animals was 20% larger than that observed in the animals treated with the GSNO-containing hydrogel.

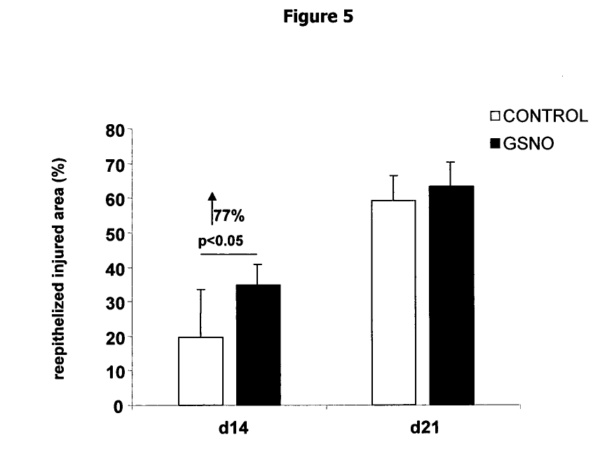

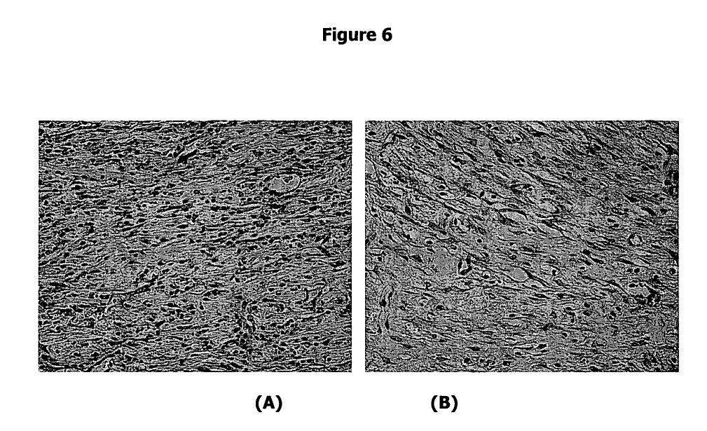

FIG. 5 shows that 7 days after injury, the area of wound reepithelization was larger in the group treated with GSNO incorporated to the hydrogel (>77%) compared to the control group. Twenty-one days after injury, a larger number of inflammatory cells were observed in superficial and deep areas of the granulation tissue in the control group, in comparison to the group treated with the GSNO-containing hydrogel. In addition, there was an increase in the number of fibroblasts in superficial and deep areas of the granulation tissue, compared to the control group. Theses cells presented as fusiform cells arranged parallel to the surface (FIG. 6).

Twenty-one days after injury, in the control group, yellow-reddish collagen fibers were observed in superficial and deep areas of the granulation tissue. In addition, in some regions of the control group, collagen fiber distribution was perpendicular to the superficial area of the group treated with GSNO, with presence of collagen fibers (thin yellow-greenish fibers) and red-yellowish fibers arranged parallel to the surface. In deep areas, there was a prevalence of organized, more mature and thick collagen fibers. In addition, it could be observed that in the GSNO-treated animals there was a tendency of increase in the number of microvessels, in relation to the control group.

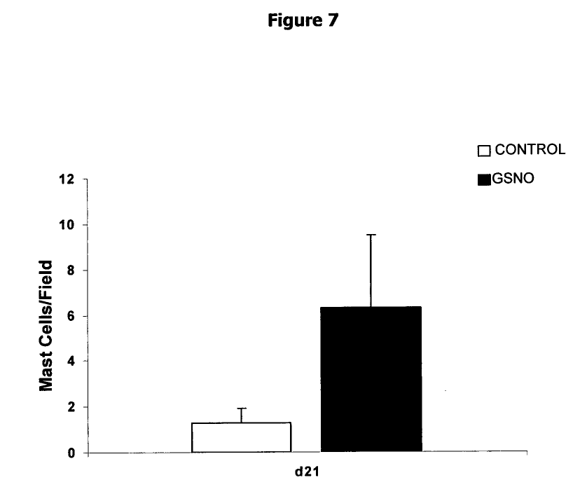

In both groups, mast cells were found mainly in deep areas of the granulation tissue, most of them with an ovoid shape and localized adjacent to the blood vessels. Twenty-one days after injury, the total number of mast cells in deep areas of the granulation tissue was larger in the GSNO-treated animals (+384%), compared to the control group (FIG. 7).

Several cell types, such as inflammatory cells, fibroblasts, endothelial cells and keratinocytes, are involved in cutaneous wound healing. Mast cells are among these cells and are important in cutaneous wound healing because they are capable of regulating the inflammatory cell migration and the formation of granulation tissue by control of angiogenesis and fibroblastic proliferation, and NO synthesis.

These results showed that a topical application of GSNO hydrogel during the first phases of the cutaneous wound healing process accelerates wound closure and its reepithelization, improves granular tissue organization, accelerates the inflammatory phase, increases the number of collagen fibers and its organization and increases the number of mast cells.

1-29. (canceled)

30. A topical S-nitrosothiol-based pharmaceutical product characterized by comprising a multiple compartment device constituted by a reaction compartment containing an acid aqueous solution; one storage compartment containing a mixture of a nitrosable thiol, or an acid salt of the nitrosable thiol, and a nitrite salt, both in the solid form, or optionally, two storage compartments to enclose separately a nitrosable thiol in the solid form and a nitrite salt in the solid form; and a formulation compartment containing a hydrophilic macromolecular composition, which may be either coupled to or uncoupled from the reaction compartment, and wherein a topical pharmaceutical composition comprising a S-nitrosothiol in the form of viscous solution or hydrogel is formed upon operation of said device.

31. A topical S-nitrosothiol-based pharmaceutical product in accordance with claim 30, wherein the device comprises two storage compartments ((1) and (2)), one reaction compartment (5) and the formulation compartment is uncoupled from the reaction compartment, where the two storage compartments (1) and (2) have a sharp format in their open end to break the bases of the components (3) and (4); and the storage compartments (1) and (2) are wrapped by components (3) and (4) that have a cup-shaped format.

32. A topical S-nitrosothiol-based pharmaceutical product in accordance with claim 31, wherein the reaction compartment (5) has two openings closed by the components (3) and (4) that permits the communication between the compounds enclosed in the storage compartments (1) and (2) and the reaction compartment (5) compounds.

33. A topical S-nitrosothiol-based pharmaceutical product in accordance with claim 31, wherein the compartments (1) and (2) can be pressed simultaneously to open the reaction compartment (5) openings.

34. A topical S-nitrosothiol-based pharmaceutical product in accordance with claim 30, wherein the device comprises four parts (6, 7, 8 and 9) and three compartments (10, 11 and 12) where the compartment 11 is both the storage and reaction compartment; compartments 10 and 12 are the storage and formulation compartments, respectively

35. A topical S-nitrosothiol-based pharmaceutical product in accordance with claim 34 wherein the part 6 is coupled to part 8 by means of a notch that allows the part 6 rotates freely over part 8.

36. A topical S-nitrosothiol-based pharmaceutical product in accordance with claim 34 wherein the part 7 consists of a t-shaped piston with an upper screw threaded to part 6.

37. A topical S-nitrosothiol-based pharmaceutical product according to claim 30, characterized by the fact that the nitrosable thiol is a mixture of nitrosable thiols or a single thiol.

38. A topical S-nitrosothiol-based pharmaceutical product according to claim 30, characterized by the fact that the nitrosable thiol is an amino acid, a peptide, a protein or any other molecule containing one or more sulfhydryl groups (—SH) in its structure.

39. A topical S-nitrosothiol-based pharmaceutical product according to claim 30, characterized by the fact that the nitrosable thiol is selected from the group consisting of glutathione (GSH), N-acetyl-cysteine (NAC) and —N-acetylpenicillamine.

40. A topical S-nitrosothiol-based pharmaceutical product according to claim 30, characterized by the fact that the nitrosable thiol and nitrite salts are present in equimolar amounts, or optionally, the amount of nitrite salt is in excess in relation to the molar quantity of the nitrosable thiol.

41. A topical S-nitrosothiol-based pharmaceutical product according to claim 30, characterized by the fact that the acid aqueous solution is present in a sufficient amount to dissolve the nitrosable thiol and the nitrite salt, according to their solubility, and yield the synthesis of S-nitrosothiol within the 1 to 6 pH range.

42. A topical S-nitrosothiol-based pharmaceutical product according to claim 30, characterized by the fact that the reaction compartment alternatively encloses water in a sufficient amount to dissolve the acid salt of the nitrosable thiol and the nitrite salt, according to their solubility, and yield the synthesis of S-nitrosothiol in the 1 to 6 pH range.

43. A topical S-nitrosothiol-based pharmaceutical product according to claim 30, characterized by the fact that the nitrite salt is sodium nitrite and the acid aqueous solution is a hydrochloric acid solution 1-4 mol L⁻¹ or a citric acid solution 1-4 mol L⁻¹.

44. A topical S-nitrosothiol-based pharmaceutical product according to claim 30 characterized by the fact that the hydrophilic macromolecular composition is constituted of one or more biocompatible hydrophilic macromolecular components.

45. A topical S-nitrosothiol-based pharmaceutical product according to claim 44, characterized by the fact that the hydrophilic macromolecular composition comprises the triblock copolymer of poly(ethylene glycol)-poly(propylene glycol)-poly(ethylene glycol) (PEO-PPO-PEO).

46. A topical S-nitrosothiol-based pharmaceutical product according to claim 44, characterized by the fact that the hydrophilic macromolecular composition comprises hydroxyethyl cellulose (HEC).

47. A topical S-nitrosothiol-based pharmaceutical product according to claim 44, characterized by the fact that the hydrophilic macromolecular composition comprises polymers of acrylic acid cross-linked with polyalkenyl ethers or divinyl glycol (Carbopol®).

48. A topical S-nitrosothiol-based pharmaceutical product according to claim 44, characterized by the fact that the hydrophilic macromolecular composition comprises poly(vinyl alcohol).

49. A topical S-nitrosothiol-based pharmaceutical product according to claim 30, characterized by containing in one of its storage and/or formulation compartments one or more agents selected from the group consisting of conserving, buffering, colorant, dispersing agents, metal complexants, and mixtures thereof.

50. A topical S-nitrosothiol-based pharmaceutical product according to claim 49, characterized by the fact that the dispersing agent is mannitol and/or a solid organic acid, such as citric acid.

51. A process for extemporaneous synthesis and incorporation of S-nitrosothiol in a hydrophilic macromolecular composition by means of the operation of the device of claim 30 topical S-nitrosothiol-based pharmaceutical product, characterized by comprising the steps of:
(a) performing a first mechanical action of said device that promotes the contact of the nitrosable thiol and nitrite salt, both in solid form, deriving from different storage compartments or from the same storage compartment of said device, with the acid aqueous solution enclosed in the reaction compartment of said device, thus forming an S-nitrosothiol through an immediate S-nitrosation reaction; and
(b) performing a second mechanical or transference action of said device, promoting the incorporation of the freshly synthesized S-nitrosothiol to the hydrophilic macromolecular composition enclosed in the formulation compartment of said device,
thus resulting in a topical pharmaceutical composition in the form of viscous solution or hydrogel.

52. Process according to claim 51, characterized by the fact that the nitrosable thiol and nitrite salts are used in equimolar amounts, or optionally, the amount of nitrite salt is in excess in relation to the molar quantity of the nitrosable thiol.

53. Process according to claim 51, characterized by the fact that the acid aqueous solution is employed in a sufficient amount to dissolve the nitrosable thiol and the nitrite salt, according to its solubility, and yield the synthesis of S-nitrosothiol within the 1 to 6 pH range.

54. Process according to claim 51, characterized by the fact that alternatively employs water in a sufficient amount to dissolve the acid salt of the nitrosable thiol and the nitrite salt, according to their solubility, and yield the synthesis of S-nitrosothiol in the 1 to 6 pH range.

55. Process according to claim 51, characterized by the fact that the nitrite salt is sodium nitrite and the acid aqueous solution is a hydrochloric acid solution 1-4 mol L⁻¹ or a citric acid solution 1-4 mol L⁻¹.

56. Process according to claim 51, characterized by the fact that the macromolecular components are already cross-linked or undergo cross-linking by the action of a cross-linking agent.

57. Topical pharmaceutical composition characterized by comprising a fleshly synthesized S-nitrosothiols incorporated in a hydrophilic macromolecular compositions selected from the group consisting of triblock copolymer of poly(ethylene glycol)-poly(propylene glycol)-poly(ethylene glycol) (PEO-PPO-PEO), hydroxyethyl cellulose (HEC), crosslinked acrylic acid-based polyalkenyl polyether (Carbopol), and poly(vinyl alcohol) presented as a viscous liquid solution or as a hydrogel obtained by the process of claim 51.

58. Use of the formulation incorporating a S-nitrosothiol resulting from the operation of the device of claim 30 topical S-nitrosothiol-based pharmaceutical product for the manufacture of a medicament for stimulation of blood flow, blood vessel dilatation, treatment of vascular insufficiencies, treatment of Raynaud's syndrome, modification of skin pigmentation, promotion and acceleration of skin, muscle, tendon, ligament, mucosa, bone and corneal wound healing, prevention of necrosis, treatment of eczemas and arthritis, systemic lupus erythematosus and cutaneous leishmaniasis.
